(19) United States
(12) Patent Application Publication
(10) Pub. No.: US 2020/0294664 A1
(43) Pub. Date: Sep. 17, 2020

(54) ADDING NEW ELECTRONIC EVENTS INTO AN ELECTRONIC USER PROFILE USING A LANGUAGE-INDEPENDENT DATA FORMAT

(71) Applicant: Babylon Partners Limited, London (GB)

(72) Inventors: Georgios STOILOS, London (GB); Domenico CORAPI, London (GB); Hugh SIMPSON, London (GB); Forat LATIF, London (GB); Chun Lok LING, London (GB); Szymon WARTAK, London (GB); Samuel WRIGHT, London (GB); Mohammad KHODADADI, London (GB)

(21) Appl. No.: 16/353,816

(22) Filed: Mar. 14, 2019

(51) Int. Cl. G16H 50/20; G16H 80/00; G06N 5/02; H04L 29/08; H04L 12/58; G06N 5/04

(52) U.S. Cl. CPC G16H 50/20; G16H 80/00; G06N 5/02; G06N 5/04; H04L 51/02; H04L 67/22; H04L 67/306

(57) ABSTRACT

The present disclosure provides a computer-implemented method of graphically representing events relating to a plurality of users. The method comprises: graphically representing a knowledge base, the knowledge base comprising concepts that are linked by relations; receiving a plurality of interim graphs each relating to an event, said interim graphs each comprising a plurality of nodes including a node identifying the user associated with the event and a node identifying a concept describing an outcome of the event; linking the plurality of interim graphs with the knowledge base to form a relation between the nodes in the interim graphs identifying the concepts and corresponding concepts in the knowledge base to produce a graphical representation of a user profile including the knowledge base augmented with the interim graphs relating to a plurality of users.

### FIELD

Embodiments described herein relate to methods and systems for generating a user profile. The user profile may be in a diagnostic system.

### BACKGROUND

A diagnostic system may include a knowledge base including medical concepts, a statistical inference engine, and a chatbot for interfacing with a user in order to diagnose a user's condition using the medical concepts from the chatbot. The chatbot may generate one or more concepts from the consultation. The concepts may be encoded using, for example, XML and sent to a database for storage. Upon request from a medical practitioner, the concepts may be retrieved from the database to build a user profile to analyse the clinical history of a patient after a number of consultations.

### BRIEF DESCRIPTION OF THE FIGURES

The present disclosure is best described with reference to the accompanying figures, in which:

FIG. 1 shows a block diagram of the diagnostic system;

FIG. 2 shows a computer for implementing the diagnostic system from FIG. 1;

FIG. 3 shows a method of generating a user profile for the diagnostic system from FIG. 1, using the computer from FIG. 2;

FIG. 4 shows a method of generating a user profile for the diagnostic system from FIG. 1, using the computer from FIG. 2;

FIG. 5 shows a method of generating a user profile for the diagnostic system from FIG. 1, using the computer from FIG. 2;

FIG. 6 shows the user profile in the form of a user graph;

FIG. 7 shows the user profile in the form of a table;

FIG. 8 shows a method of generating a user history from the diagnostic system from FIG. 1, using the computer from FIG. 2; and

FIG. 9 shows a method of generating a user history from the diagnostic system from FIG. 1, using the computer from FIG. 2.

### DETAILED DESCRIPTION OF THE EMBODIMENTS

Embodiments of the present disclosure relate to a computer-implemented method of building a user profile for a medical diagnostic system. The method comprises: receiving a new event including data describing a chatbot consultation with the user; encoding the new event using JSON; storing the encoded new event in a queue of events; decoding and translating the new event into a form compatible with the user profile; and adding the translated new event to the user profile.

It is an object of the present disclosure to improve on the prior art. In particular, the present disclosure addresses a technical problem tied to computer technology and arising in the realm of computer networks, namely the technical problem of bandwidth usage and processing speed. The disclosed system solves this technical problem using a technical solution, namely by encoding events from a chatbot consultation using JSON and storing the encoded event in a queue for subsequent retrieval, decoding and translation into a user profile. JSON provides a standardised format for concept encoding requiring reduced bandwidth for transmission, and using the queue allows for the user profile to be built up incrementally saving processing each time the user profile is generated.

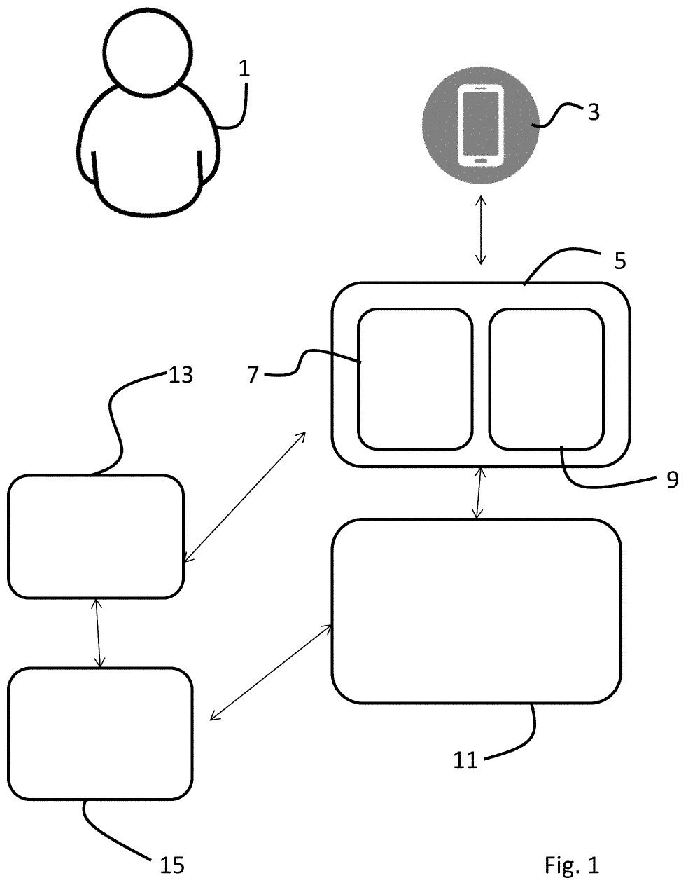

With reference to FIG. 1, a user 1 communicates to a diagnostic system via a mobile phone 3. However, any device could be used, which is capable of communicating information over a computer network, for example, a laptop, tablet computer, information point, fixed computer, voice assistant, etc.

The mobile phone 3 will communicate with interface 5. Interface 5 has two primary functions, the first function 7 is to take the words uttered by the user and turn them into a form that can be understood by the inference engine 11. The second function 9 is to take the output of the inference engine 11 and to send this back to the user's mobile phone 3.

In some embodiments, Natural Language Processing (NLP) is used in the interface 5. NLP is one of the tools used to interpret, understand, and then use everyday human language and language patterns. It breaks both speech and text down into shorter components and interprets these more manageable blocks to understand what each individual component means and how it contributes to the overall meaning, linking the occurrence of medical terms to the knowledge base. Through NLP it is possible to transcribe consultations, summarise clinical records and chat with users in a more natural, human way.

However, simply understanding how users express their symptoms and risk factors is not enough to identity and provide reasons about the underlying set of diseases. For this, the inference engine 11 is used. The inference engine 11 is a powerful set of machine learning systems, capable of reasoning on a space of >100s of billions of combinations of symptoms, diseases and risk factors, per second, to suggest possible underlying conditions. The inference engine 11 can provide reasoning efficiently, at scale, to bring healthcare to millions.

In an embodiment, a knowledge base 13 is a large structured set of data defining a medical knowledge base. The knowledge base 13 describes an ontology, which in this case relates to the medical field. It captures human knowledge on modern medicine encoded for machines. This is used to allow the above components to speak to each other. The knowledge base 13 keeps track of the meaning behind medical terminology across different medical systems and different languages. In particular, the knowledge base 13 includes data patterns describing a plurality of semantic triples, each including a medical related subject, a medical related object, and a relation linking the subject and the object. An example use of the knowledge base would be in automatic diagnostics, where the user 1, via mobile device 3, inputs symptoms they are currently experiencing, and the interface engine 11 identifies possible causes of the symptoms using the semantic triples from the knowledge base 13. The subject-matter of this disclosure relate to creating and enhancing the knowledge base 13 based on information described in unstructured text.

A user graph 15 is also provided and linked to the knowledge base as discussed in more detail below.

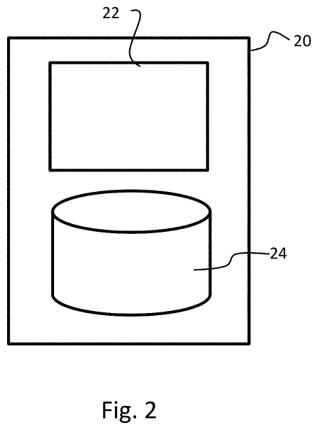

With reference to FIG. 2, a computer 20 is provided to enable the interface engine 11 and the knowledge base 13 (from FIG. 1) to operate. The computer 20 includes a processor 22 and a memory 24. The memory 24 may include a non-transitory computer readable medium for storing electronic data. The memory 24 may refer to permanent storage. The electronic data may include instructions which, when executed by the processor cause the processor to perform one or more of the methods described herein.

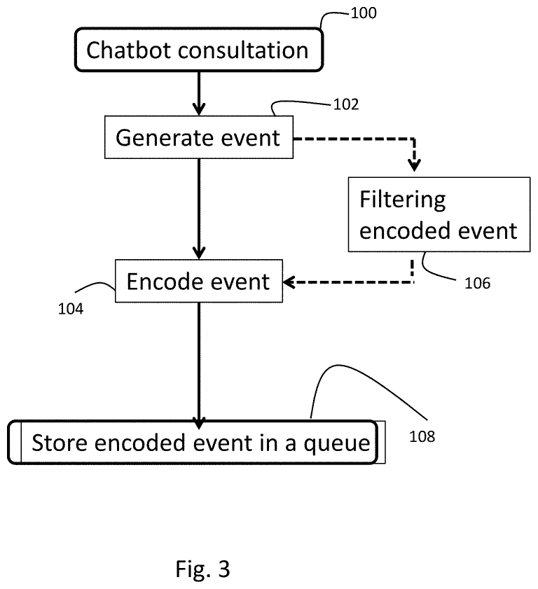

With reference to FIG. 3, a patient, e.g. Adam, goes through a consultation using a conversation module of the diagnostic system at step 100. The conversation module is part of the interface engine 11 and may be provided in the form of a chatbot. The chatbot is a computer program implemented by the interface 5 (FIG. 1). The chatbot is able to pose questions to the patient and interpret the responses. In particular, the questions may be based on semantic triples contained in the knowledge base 13 or the inference engine (FIG. 1). The responses may be used to derive a diagnosis, and a concept may be generated to describe the diagnosis.

At step 102, an event is generated to describe the consultation. The event may include one or more concepts describing symptoms as well as the diagnoses (as described in more detail below). The event may also include event information such as an event identifier (ID), a user ID, a time stamp at which the consultation took place, and a location of the consultation. The user ID may be determined from the IP address of the user device. The time stamp may be obtained from the clock of the user device. The location of the consultation may be obtained from a positioning system of the user device, e.g. a global positioning system (GPS). The event information may be included in the form of metadata.

At step 104, the event is encoded as described below.

During the chatbot consultation, the patient may input “I have an acute pain in my left leg”, which contains the complex medical notion “Acute pain in left leg” that needs to be identified and encoded in a formal way using concepts from the medical knowledge base 13 (FIG. 1). The chatbot outputs a concept to describe the complex medical notion. For instance, given the above sentence, the following concept may be generated:

Pain [symbol] ∃hasQualifier .Acute [symbol] ∃findingSite.LeftLowerLimb

where Pain, Acute, and LeftLowerLimb are concepts from the knowledge base 13 and hasQualifier and findingSite are properties (binary relations).

The concept above is written in (abstract) description logic (DL) syntax and in order to be transmitted between different computer systems, or even saved in a data store, it needs to be serialised into a machine readable format.

Various different services within the diagnostic system may need to exchange concepts between them. For example, an engine for triaging may need to ask the user further questions about their reported symptoms in order to proceed with the symptom checking process or retrieve sonic information from the user graph 15. The answers of questions also represent complex medical notions. For example, for a user reporting an injury in his hand, the symptom checking system may need to ask the following question:
“Ouch! So do you have any of the following?”with potential answers being:
   1. “A bleeding wound”
   2. “An animal or human bite that has broken the skin”
   3. “A crooked finger, thumb or hand”

The user friendly text above is associated with a concept that captures its meaning. Answers are captured through a (complex) medical concept which is built using other concepts, where each concept is described in association with an international resource identifier (IRI) from the medical knowledge base 13. For example, answer 1 corresponds to the complex concept
Wound [symbol] ∃associatedWith.Bleeding

whereas answer 2 corresponds to complex concept
BrokenSkin [symbol] ∃dueTo.(BiteOfAnimal ␣ BiteOfHuman).

These complex concepts need to be transmitted to the user together with the respective user friendly text to be rendered. In addition, the final diagnosis for a patient, the reported symptoms and any other related condition, which are again represented as complex medical concepts need to be stored in his/her profile. Besides the aforementioned ones, several other services within the diagnostic system generate, store or exchange complex medical knowledge for users/patients.

Thus, it becomes apparent that serialising and transmitting concepts between these services is of paramount importance in order for the services to intercommunicate, coordinate, and interoperate. The format needs to be simple, compact, easy to serialise/deserialise, and transmit over the network, as well as comprehensible by software engineers and medical personnel that are developing the services.

An encoding format only specifies some general rules for creating concepts. The freedom of the encoding rules make it possible for services (and actually even humans) to create erroneous concepts or simply concepts of low quality. For example, the following concept is of low-quality in the sense that it is an empty concept (does not represent anything in the real-world).
Wound [symbol] Bleeding

This is the case because the two concepts Wound and Bleeding are of different semantic type (they are not a like for a like) and hence their intersection (conjunction) is empty. Although this example is quite obvious there are more involved examples like
Person [symbol] ∃treatedBy.Malaria
   The above concept is structurally correct however semantically it is empty.

In order to achieve high levels of interoperability, quality, and reduce the number of empty concepts an additional set of constraints are required that can be used to eliminate or prevent the generation of such concepts. In order to do this, the diagnostic system uses a JavaScript Object Notation (JSON)-based format for serialising and exchanging concepts. The use of JSON makes the format easy to process and exchange as JSON is one of the most popular formats for exchanging data between web services.

Concepts used by the diagnostic system may be defined by the following Backus-Naur Form (BNF) syntax:

binaryRel := “and” | “or” | “neither”
unaryRel := “not” | “unknown”
Datatype := (integer|member|string|date)
Concept := (NullConcept|SimpleConcept|UnaryConcept|BinaryConcept|ModifiedConcept)
SimpleProperty := (“label”: String, “iri”: propertyIRI)
Property := (SimpleProperty| ( “not” : SimpleProperty )
NullConcept := ( )
SimpleConcept := (“label”: String, “iri”: conceptIRI)
UnaryConcept := ( unaryRel : Concept )
BinaryConcept := ( binaryRel : ( Concept ) )
ModifiedConcept : = ( “baseConcept” : BCType, “modifiers” : [ Modifier+ ] )
Modifier := { “type” : Property, “value” : (Concept | ValueUnitConcept | RangeConcept) }
ValueUnitConcept := (“value”: string, “valueType”: Datatype, “unit”:Concept)
RangeConcept := (“min”: ValueUnitConcept|NullConcept, “max”; ValueUnitConcept|NullConcept)
SimpleUnaryConcept := ( unaryRel : SimpleConcept )
BCType := (SimpleConcept|SimpleBin|SimpleUnaryConcept)
SimpleBin := ( binaryRel : [SimpleConcept+ ] )

A modified concept can be encoded in JSON as follows. For an example concepts of “a bleeding wound”, the encoded form in JSON would be:

{

“baseConcept”: {

“iri”: https://bbl.health/ud_nQ1D6Sx”,
“label”: “wound”

},
“modifiers”: [
{

“type”: {

“iri”: “ https://bbl.health/CowWSKjAdo”,
“label”: “associated with”

},
“value”: {

“iri”: “ https://bbl.health/5YbSWtY38M”,
“label”: “bleeding”

} } ] }

The JSON-format is a syntax and does not provide with formal semantics of constructs, structural restrictions on concepts, or a deeper insight on the complexity or the properties of the concepts that can be constructed using it. To do so, it is good to try and map this syntax also to a formal language like that of Description Logic. Table 1 below presents a mapping from the JSON-constructs defined above to the corresponding DL notation.

| | |
|---|---|
| SimpleProperty | R |
| { “not” : SimpleProperty } | ¬R |
| NullConcept | ⊥ |
| SimpleConcept | A |
| {“not”: Concept} | ¬C |
| {“unknown”: Concept} | U C |
| { binRel: [C1, . . . , Cn] } where n ≥ 2 | C1 binRel . . . binRel Cn where |
| | binRel ∈ {Π, ␣} |
| {baseCon: BCType, “modifiers”: | BCType Π;i ∃propertyIRIi.Ci |
| [mod_1, . . . , mod_m]} | |
| where each mod_i is of the form | |
| { “type” : propertyIRI_i, “value” : C_i } | |
| and m>0 | |
| {“value”: num, “valueType”: type, | value {num\hat{ } type : Con} where type |
| “unit”:Con} | is one of the known datatypes |
| | int, number, string, date |
| {“min”: ValueUnitConcept \| | [value1, value2], [⊥, value2], [value1,⊥] |
| NullConcept, “max”: | |
| ValueUnitConcept \| NullConcept} | |

The above translation can produce concepts of the form E [symbol] ∃-R.D. Semantically, these concepts are implying concepts of the form E [symbol] ¬∃R.D. Summarising, the complex concepts constructed using the JSON-syntax presented above roughly correspond to DL concepts constructed using the following syntax.

| | | |
|---|---|---|
| C, D | → | ⊥ \| A \| ¬C \|CΠD \| C␣D \| EΠ∃R.D \| UC |
| | | ∃hasQuantifier.{num\hat{ }type : Con} \| |
| | | ∃P.[num1 \hat{ }type: C1, num2\hat{ }type: C2 ] |
| | | and P is some subPropertyOf hasQualifier |
| E | → | A1 Π A2 Π. . . Π An \| A1 ␣ A2 ␣ . . . ␣ An \| ¬A |

U is an operator called “unknown”. It is a non-standard operator in Description Logic but its semantics can be given using some 3-valued logic where UC obtains the truth value of 0.5.

Example semantics for unknown concepts are the following:
{UA} ∪ K [symbol] UB for every K [symbol] B [symbol] A

which follows Lukasiewicz logic in the sense that the proposition “unknown implies unknown” is true. Hence, if K [symbol] B [symbol] A and A is unknown that B is implied to be unknown. In contrast, in Kleen logic (min-max logic) “unknown implies unknown” is unknown and “false implies unknown” is true.

Consequently, with the Lukasiewicz logic-based semantics if some concept is set to “unknown” then all sub-concepts of it in the Knowledge Base are implied to by “unknown” as well.

The mapping to Description Logics presented above can help us into developing a set of constraints that can be used for ensuring the quality and coherency of complex concepts created using the JSON format. In the following we present a definition of the current constraints implemented as a validation service for complex concepts. Some of these constraints are implemented with the help of the Knowledge Base and some upper-level-model encoded in the Knowledge Base. This upper-level-model describes some constraints on the acceptable models.
Definition. For a set S of concepts of the form {C1, . . . , Cm} we use the notation [symbol] S to denote the conjunction of the form C1 [symbol] . . . [symbol] Cm; dually with [symbol] S. Let K be a Knowledge Base, KB. The domain (resp. range) for a property R is a set Δ (resp. P) of concepts from K such that for every triple <s R o>, there is some d∈Δ such that K [symbol] s [symbol] d. The range for a property R is a set P of concepts from K such that for every triple <s R o>, there is some r∈P such that K [symbol] o [symbol] r.

Definition. Let K be some KB and let δ be mappings from properties in a KB to their domain. Similarly, let ρ be mappings from properties in a KB to their range. Let also sty be a set of concepts from K called semantic types. A complex concept is well-formed if the following conditions hold:
1. All concepts in a binary concept must have some common semantic type.
2. In modified concepts of the form E [symbol] ∃R.D., we should have K [symbol] [symbol] ␣δ(R) and K [symbol] D [symbol] ␣ρ(R), i.e., the base(resp value) of the modifier should be a descendant of some of the domain (resp. ranges) of the property.
3. For range concepts the units used in the “min” and “max” need to be of “compatible” semantic types. By compatible we mean of compatible sorts or of unit systems that can be translated from one to the other. In other words, a range concept should have minimum and maximum values that do not have inconvertible units. For example, we can have unit “kilograms” for “min” and unit “pounds” for “max” but not “Months”. This restriction is not easy to implement but can be of the form: “both should have a common parent class in the KB”.
4. RangeConcepts should always be under the scope of some modifier.
5. “Has Quantifier” should be the only property that is used to link modifier values that are of type ValueUnitConcept.
6. Units used in ValueUnitConcept should be descendants of Unit by category (https://bbl.health/dMibjj2-Wx) or descendants of SI units (https://bbl.health/DJ_XQSBZmQ).

At step 106, the concepts may be filtered according to the criteria numbered 1-6 above. In this way, only concepts that fulfil the above criteria are encoded, and so only concepts of sufficient quality are encoded to reduce the overall number of concepts being encoded such that processing burden is reduced. Step 106 is shown as a broken line as it is optional.

At step 108, the encoded event is stored in a queue of events. The other events in the queue may include other events that have previously been obtained from the chatbot for the same user. The queue of events is stored as electronic data in the memory 24 (FIG. 2). Such recording of events is in partial fulfilment of the architectural pattern known as event sourcing.

Once a queue of events is available for a user, a user profile can be built as a projection by a projector. The user profile can take the form of a user graph or a table of information specific to the patient.

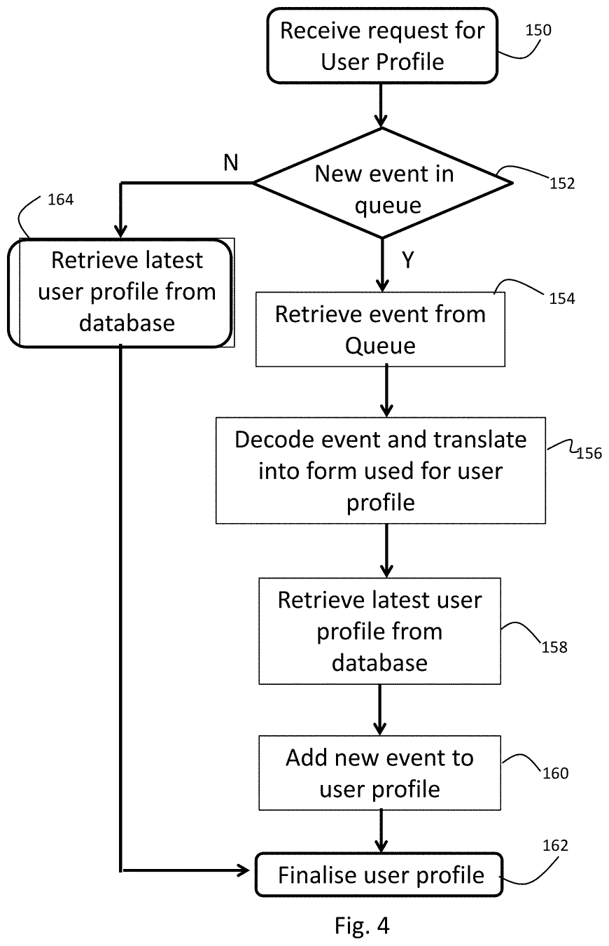

With reference to FIG. 4 a user profile request is received at step 150. The user profile request may be received as a manual user request through user interface 5 (FIG. 1).

At step 152, the queue is checked to determine if a new event has been recorded since the previous iteration of the user profile.

If there has been a new event recorded since the previous iteration of the user profile the new event is retrieved from the queue at step 154.

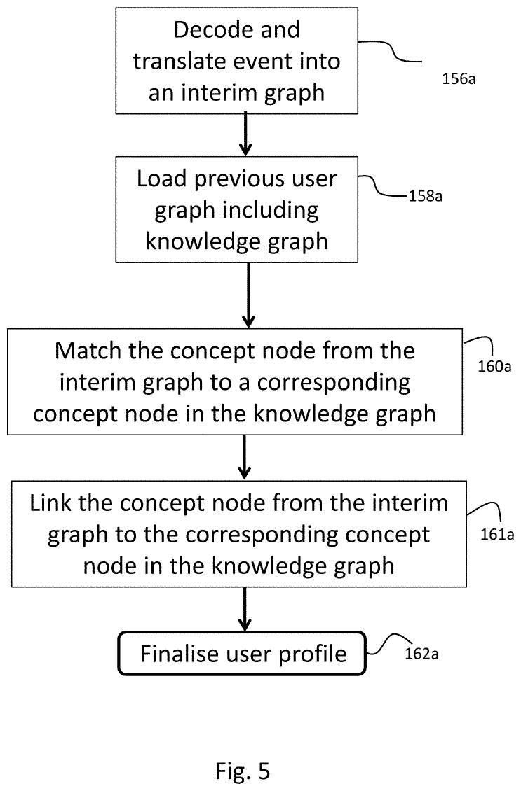

At step 156, the event is decoded from the format used to store the event in the queue. In particular, the event is decoded from the JSON format. The decoded event is translated into a form used for the user profile. Where the user profile is a user graph, the event is translated into a set of nodes and edges linking the nodes, as described below in relation to FIG. 5.

At step 158, the latest version/iteration of the user profile is retrieved from the memory 24 (FIG. 2). Next, the new event is added to the user profile at step 160. Finally, the user profile is finalised at step 162, and stored as the latest iteration of the user profile in the memory 24 (FIG. 2). For instance, the user profile may be transmitted to the user interface 5 (FIG. 1).

In the event that there is no new event in the queue, the latest iteration of the user profile is retrieved from the memory 24 (FIG. 2) at step 164 and used as the user profile.

FIG. 5 shows the specific case of generating a user profile in the form of a user graph, and follows the steps outlined in FIG. 4, together with more detail as recited below.

At step 156a, the event is decoded and translated into an interim graph. The interim graph includes a plurality of nodes and edges linking the nodes. The nodes represent information from the event. For instance, one node corresponds to an event identifier (ID), one node may correspond to a concept derived from the chatbot consultation (e.g. the concept may define the diagnosis), one node may correspond to a time stamp associated with the consultation, one node may correspond to a location of the user during the consultation, and one node may correspond to an identifier of the user.

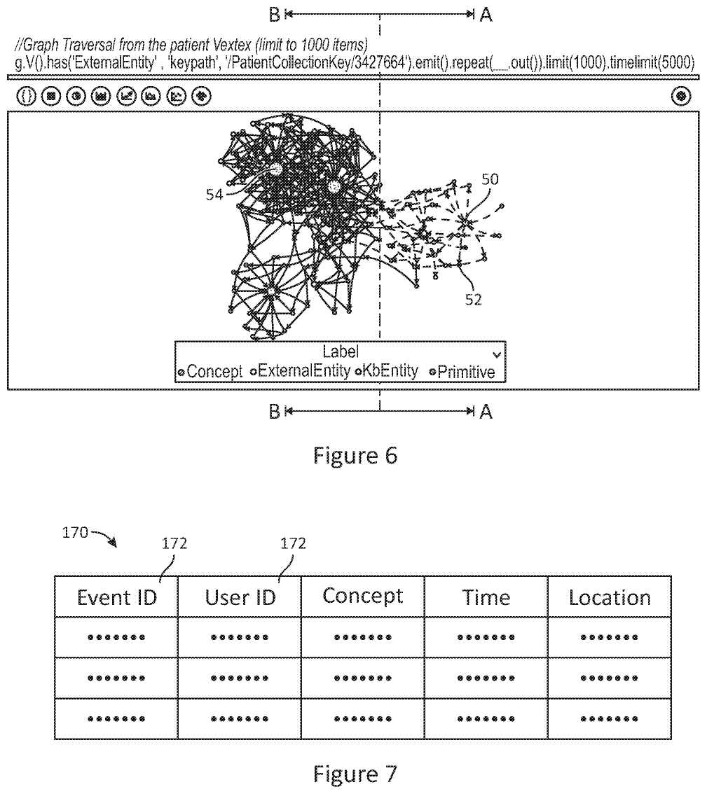

At step 158a, the previous version of the user graph is retrieved from the memory 24 (FIG. 2). As shown in FIG. 6, the user graph 15 includes the knowledge graph 13. As described above, the knowledge graph includes nodes defining medical concepts, and edges linking the medical concepts. For instance, each node may represent an element <subject, property, object> of a semantic triple derived from unstructured text. The edges may link related elements.

As shown in FIG. 6, the event identifiers are shown as nodes 50, and the other information from an event are shown as nodes 52, all of which are represented in section A. The knowledge base concepts are shown as nodes 54, and are represented in section B.

The other information of an event shown as nodes 52 in FIG. 6 may include the other information listed above. The term “user profile” may be taken to mean that the user profile corresponds only to a single user of the chatbot. In this case, the nodes 52 representing a user ID are all the same in the user profile. In other cases, the user profile may be taken to mean all user data collected from the chatbot irrespective of user. For instance, a plurality of users may each carry out diagnoses using the chatbot. Each of consultation will be added to the user profile and so the nodes 52 representing user IDs may be different by relating to a plurality of users.

With further reference to FIG. 5, at step 160a, the interim graph is added to the user graph. The concept from the interim graph is matched to a concept of the knowledge graph.

At step 161a, the nodes of the interim graph and the knowledge graph corresponding to the matched concepts are linked using an edge. In this way, the newly added interim graph is joined to the knowledge graph and so is integrated within the user graph.

At step 162a, the user profile is finalised. The user profile may be transmitted to the memory 24 (FIG. 2) to store as electronic data. The user profile may also be transmitted to the user interface 11 (FIG. 1).

With reference to FIG. 7, the user profile may also take the form of a table 170. The table includes headings 172 representing the information from the event. The headings are arranged in columns and include the event identifier (ID), the user identifier (ID), the concept, and time, and the location. The rows correspond to the individual events, e.g. information for a single chatbot consultation is included in a row. When data from a new event is added to the user profile (step 160 from FIG. 4), the new data is arranged according to the different headings. In this way, the event information is converted to structured data in the user profile.

Once the user profile is available in either form (e.g. user graph or table), a user (e.g. a medical professional) can request analytics.

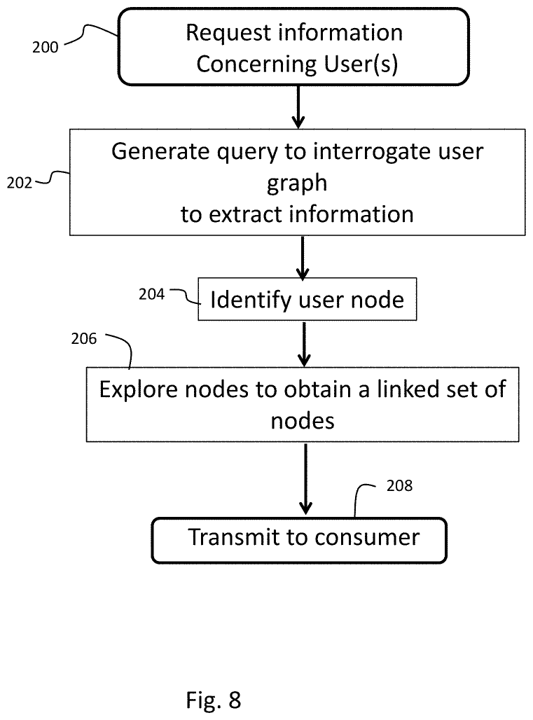

With reference to FIG. 8, a user may request, via the interface 5 (FIG. 1), information to be extracted concerning one or more users step 200.

A query is generated by the interface engine 11 (FIG. 1) and includes the user identifier and a concept relating to the condition of interest at step 202. The user identifier may be a single user identifier in the event that a single user history is requested, or may include a plurality of user identifiers (IDs) in the event that multiple user histories are requested. The number of user IDs involved in the query may be any number from one to all of the users known to the system. The interface 11 (FIG. 1) retrieves the user graph from the memory 24 (FIG. 2). The user node(s) is identified using the user identifier from the query at step 204. The query may also include a concept of interest, e.g. dementia. The concepts may include subclasses of the concept from the query (e.g. the concept is dementia and the subclass is senile dementia). The search may be carried out for a pre-determined branch factor, and depth, to limit the number of edges that the search traverses. The obtained concepts may also include risk factors linked to the condition, e.g. being a smoker where the condition is dementia. The nodes traversed during the search are compiled in a user history. The user history thus may include a plurality of concepts linked to the user. As indicated above, the user graph may relate to a single user or to a plurality of users. In the event of a plurality of users, the query may be sent iteratively for each user in a list of users.

Once the user history has been compiled, the concept in the query may be used to filter the extracted information from the user history. For instance, where the concept relates to dementia, and where all of the users have been included in the query, several users may have no history of dementia. Accordingly, the filtering will return to the user interface, at step 208, only information relating to users where dementia is included for them in the user history.

In this way, the requesting user can obtain analytics relating to a particular condition, for a particular patient or group of patients. Such knowledge may be used to ascertain warnings relating to outbreaks of certain conditions, for example, a new strain of flu for users in a particular area. For example, the query may include a reference to a geographical region, and cover all users within that region, together with the condition or symptoms. In this way, the user nodes will be identified by identifying all user nodes linked to a node 52 (FIG. 6) representing the geographical region within a defined range set by the requesting user.

When obtaining the concepts at step 206, the projector extracts the IRI of each identified concept. For instance, for the following event:

{

“patientId”: “3427664”,
“concept”: {

“baseConcept”: {

“label”: “Long-term drug therapy”,
“iri”: “266713003”

},
“modifiers”: [

{

“type”: {

“label”: “USING SUBSTANCE”,
“iri”: “424361007”

},
“value”: {

“label”: “Non-steroidal anti-inflammatory agent”,
“iri”: “372665008”

}

}

]

}

}

the extracted IRIs are 266713003 and 372665008, which are value IRIs and are added to indexed fields in the projection. In terms of the actual implementation, they become elements in a list stored in the field entitled “all_iris”.

As a counter example, consider an architecture that does use event sourcing but does not encode the events using JSON as described above. Not having this common JSON structure enforced in events would mean that for each source it would be necessary to implement sonic ad-hoc logic in the projector (or some stream transformation) to extract the IRIs. For example, drug reports could be received as:

{

“patientId”: “3427664”

“reported_drug”: [{

duration: “long term”
substance: 372665008

}]
}

and maybe medical conditions could be reported by another system as

{

“patientId”: “3122113”

“conditions”: “headache”, “stress”
}

The projection would then have to be aware of the different structure of events and parse them differently based on the source. In this way, by encoding the events using JSON and storing each new event in a queue (event sourcing), it is possible to construct the user profile more efficiently.

The process outlined in FIG. 8 may be implemented using the following Gremlin query.

GET /clinicalgraph/path/patient/X/concept/32187
propertyIris=[‘https://bbl.health/qPyHHgsYF6’] // risk factor iri
g.V( ).has(“Key”, “keyPath”, ‘/PatientKey/’ + patient_id)

// Get all cases for a patient
.repeat(out( ))
.until(hasLabel(neq(“Key”)))
// Traverse to conditions of a patient ignoring nots (can follow
complex concepts)
.repeat(out( )

.or(

hasNot(“logicalOp”),
has(“logicalOp”, neq(“Not”)))

)

.until(hasLabel(“KbEntity”))
// Current condition is property of given in or a parent class is
property of given iri
.where(or(

inE( ).where(values(“iri”).is(within(propertyIris)))

.outV( ).has(“KbEntity”, “iri”, iri),

outE(“KnowledgeEdge”)

.where(values(“prefLabel”).is(“subClassOf”)).inV( )
.inE( ).where(values(“iri”).is(within(propertyIris)))
.outV( ).has(“KbEntity”, “iri”, in)))

.path( )

An alternative to this would be to start from a medical concept, e.g. dementia, and explore the knowledge base searching all the possible risk factors (e.g. being a smoker) and the subtypes of dementia (e.g. senile dementia). This query is not particularly complex (it's linear in the size of the graph or O(b\hat{ }d) where b is the maximum branching factor and d is the maximum depth). This would return a set of concept C to capture in the user history. Then it would be necessary to query the clinical history table (FIG. 7) and filter by the element in C. The complexity is O(u*|C|)) where u is the number of events of the given patient. This is worse compared to O(u*d) for the “graph search” as outlined in FIG. 8 (for each event to go up the is_risk_factor and subclass hierarchies).

if event sourcing wasn't being used this would be more complicated and involve additional network calls since it would be necessary to query multiple databases to aggregate all the events at query time.

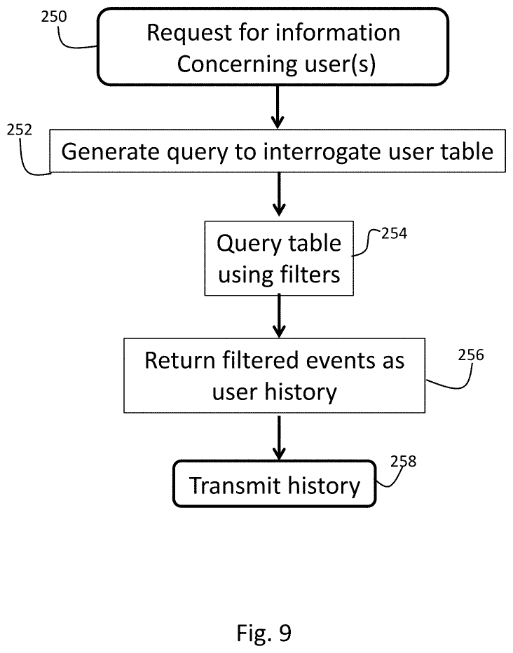

With reference to FIG. 9, the user history may be obtained instead by use of the table from FIG. 7, At step 250, the user history is requested from the interface 5 (FIG. 1). The user history may require information for a user over the past week. At step 252, a query is generated to interrogate the table. In the case mentioned above where the medical professional wishes to obtain the user's medical history over the past week, the query may include the user ID and a time stamp range.

At step 254, the interface engine 11 (FIG. 1) retrieves the table for the user from the memory 24 (FIG. 2), and filters the elements in the table using the time stamps from the query. At step 256, the interface engine 11 (FIG. 1) returns the filtered elements as the user history. The user history may be transmitted to the user interface 5 at step 258.

The process outlined in FIG. 9 can be implemented using the following code. The first command is written in http and requests a service called “Timeline”, and the second command is also written in http and requests a service called Clinicalhistory. The third command is a CQL query that Clinicalhistory performs on Cassandra.

GET /timeline/patient/X/summary?from=1540481433
v2/clinicalhistory/patients/X/clinical-records?from=1540481433
SELECT * FROM clinicalrecords where patient_id=X AND timestamp >
1540481433

Using the information for the patient over one week, various analytics can be implemented. For instance, it is possible to construct a co-occurrence matrix.

For instance, it is possible to use a “map-reduce” function to aggregate concepts by time bucket and patient. For example, all of the concepts related to the patent X, for week W, are saved into a bucket B=<X,W>. The bucket, B, may be stored as electronic data in the memory 24 (FIG. 2). Each bucket can be mapped to a symmetric matrix that has 1 for each row <c1, c2> where c1 and c2 appear in B and <c1, c1> is the number of times the condition c1 appears in the event. The matrices can be reduced to a matrix that contains the sum of all the other matrices.

The output may be:

| | headache | stress | abdominal pain |
|---|---|---|---|
| headache | 10 | 3 | 1 |
| stress | 3 | 4 | 3 |
| abdominal pain | 1 | 3 | 16 |

or normalised as:

| | headache | stress | abdominal pain |
|---|---|---|---|
| headache | 1 | 3/14 | 1/26 |
| stress | 3/14 | 1 | 3/20 |
| abdominal pain | 1/26 | 3/20 | 1 |

Features of some embodiments set out in the following clauses.
Clause 1. A computer-implemented method of building a user profile for a medical diagnostic system, the method comprising:
   receiving a new event including data describing a consultation with the user from a conversation module of the diagnostic system;
   encoding the new event using JavaScript Object Notation (JSON);
   storing the encoded new event in a queue of events;
   decoding and translating the new event into a form compatible with the user profile; and
   adding the translated new event to the user profile.
Clause 2. The computer-implemented method of Clause 1, further comprising:
   searching the queue of events for any new events in response to a request to build the user profile; and
   in response to identifying the new event in the queue of events, decoding and translating the event into a form compatible with the user profile.
Clause 3. The computer-implemented method of Clause 2, wherein the user profile is a structured table of events, wherein:
   adding the translated new event to the user profile includes assigning data of the translated new event to a plurality of headings.
Clause 4. The computer-implemented method of Clause 3, wherein the headings are selected from a list including: an event identifier, a user identifier, a time stamp of when the conversation occurred, a concept derived from the conversation, and a location of the conversation.
Clause 5. The computer-implemented method of Clause 2, wherein the user profile is a user graph, wherein:
   decoding and translating the new event into a form compatible with the user profile includes generating an interim graph including a plurality of nodes, the plurality of nodes including a node identifying the user, and a node identifying a concept derived from an outcome of the consultation.
Clause 6. The computer-implemented method of Clause 5, wherein adding the translated new event to the user profile includes:
   loading a knowledge graph including a plurality of knowledge base nodes each knowledge base node relating to a concept derived from unstructured text, and a plurality of edges, each edge linking two of the knowledge base nodes;
   matching the node from the interim graph identifying the concept derived from the outcome of the consultation with the knowledge base node identifying the closest concept to the concept derived from the outcome of the consultation; and
   linking the node from the interim graph identifying the concept derived from the outcome of the consultation with the knowledge base node identifying the closest concept to the concept derived from the outcome of the consultation.
Clause 7. The computer-implemented method of Clause 5, wherein the plurality of nodes of the interim graph also include a node identifying a location of the event, and a node identifying a time stamp of the event, and a node identifying the event.
Clause 8. A computer-implemented method of processing a concept for inclusion in a knowledge base, the method comprising:
   receiving the concept;
   encoding the concept using JavaScript Object Notation (JSON); and
   transmitting the encoded concept for inclusion in a queue of events.
Clause 9. The computer-implemented method of Clause 8, further comprising:
   in response to receiving the concept, filtering the concept based on pre-determined constraints related to a concept type.
Clause 10. The computer-implemented method of Clause 9, wherein when the concept type is of the form E [symbol] ∃ R.D, using a description logic version of the concept encoded using JSON, where E is a concept, [symbol] is a logical conjunction of two concepts, and ∃ R.D is a modifier, where ∃ is an existential operator to combine a role with a concept to form a new concept, R is a modifier type in the form of a relation, and D is a modifier value in the form of another concept, the pre-determined constraints include K [symbol] E [symbol] ␣δ(R) and K [symbol] D [symbol] ␣ρ(R), where K is a knowledge base, [symbol] denotes that something follows logically from something else, [symbol] denotes a subclass operator where one concept is a subclass of another concept, ␣ is a logical injunction of two concepts, δ represents a domain of the relation, R, and ρ(R) is a range of the relation, R.
Clause 11. The computer-implemented method of Clause 10, wherein the concept type is of the form A1 [symbol] A2 [symbol] . . . [symbol] An, using a description logic version of the concept encoded using JSON, or wherein the concept type is of the form A1 ␣ A2 ␣ . . . ␣ An, where each Ai is a concept and [symbol] is a logical conjunction of two concepts, or wherein ␣ is a logical injunction of two concepts, the pre-determined constraints include that all Ai have a common semantic type as an ancestor, as semantic type is defined in a knowledge base.
Clause 12. The computer-implemented method of Clause 10, wherein the concept type is a range concept, and wherein the predetermined constraints include both minimum and maximum values of the range concept not being inconvertible units.
Clause 13. The computer-implemented method of Clause 10, wherein the concept type is a value unit concept, and wherein the predetermined constraints include that the unit is a descendent unit of an SI unit.
Clause 14. A computer-implemented method of graphically representing events relating to a plurality of users, the method comprising:
   graphically representing a knowledge base, the knowledge base comprising concepts that are linked by relations;
   receiving a plurality of interim graphs each relating to an event, said interim graphs each comprising a plurality of nodes including a node identifying the user associated with the event and a node identifying a concept describing an outcome of the event;
   linking the plurality of interim graphs with the knowledge base to form a relation between the nodes in the interim graphs identifying the concepts and corresponding concepts in the knowledge base to produce a graphical representation of a user profile including the knowledge base augmented with the interim graphs relating to a plurality of users.
Clause 15. The computer-implemented method according to Clause 14, wherein the interim graphs each relate to a different user.
Clause 16. The computer-implemented method according to Clause 14, wherein the interim graphs are anonymised.
Clause 17. The computer-implemented method according to Clause 14, wherein the plurality of nodes also includes one or more of a node representing an event identifier, a node representing a time stamp of when the event took place, and a node representing a location of the event.
Clause 18. The computer-implemented method according to Clause 14, wherein the method further comprises:
   receiving a new event including data describing a consultation with one of the plurality of users from a conversation module of the diagnostic system;
   encoding the new event using JavaScript Object Notation (JSON);
   storing the encoded new event in a queue of events;
   decoding and translating the new event into a form compatible with the interim graph; and
   adding the translated new event to the interim graph.
Clause 19. The computer-implemented method according o Clause 18, further comprising:
   searching the queue of events for any new events in response to a request to build the user profile; and
   in response to identifying the new event in the queue of events, decoding and translating the event into a form compatible with the interim graph.
Clause 20. A computer-implemented method of extracting information concerning a plurality of users, the method comprising:
   retrieving the user profile graphically represented according to the method of Clause 14,
   receiving a query to extract information from the user profile, the information including a plurality of users,
   interrogating the user profile to identify a plurality of nodes associated with the plurality of users, and to extract information from nodes linked to the plurality of nodes associated with the plurality of users, and
   returning the extracted information for the plurality of users.
Clause 21. The computer-implemented method according to Clause 18, wherein said information concerning the plurality of users includes one or more of a concept, a location of the event, and a time stamp of the event.
Clause 22. The computer-implemented method according to Clause 18, wherein the step of returning the extracted information for the plurality of users includes filtering the extracted information to include only information relating to the query.
Clause 23. The computer-implemented method according to Clause 18, wherein interrogating the user profile to identify a plurality of nodes associated with the plurality of users includes identifying the plurality of nodes within a pre-determined branch factor.
Clause 24. A non-transitory computer-readable medium, storing instructions, that when executed by a processor, cause the processor to perform the method according to any preceding clause.

1. A computer-implemented method of graphically representing events relating to a plurality of users, the method comprising:
graphically representing a knowledge base, the knowledge base comprising nodes defining concepts, and edges linking the concepts, wherein each concept represents an element selected from a list including: a subject of a semantic triple, a property of a semantic triple, and an object of a semantic triple, the semantic triple being derived from unstructured text;
receiving a plurality of interim graphs each relating to an event, said interim graphs each comprising a plurality of nodes including a node identifying a user associated with the event and a node identifying a concept describing an outcome of the event;
linking the plurality of interim graphs with the knowledge base by forming an edge between the nodes in the interim graphs identifying the concepts and corresponding concepts in the knowledge base to produce a graphical representation of a user profile including the knowledge base augmented with the interim graphs relating to a plurality of users.

2. The computer-implemented method according to claim 1, wherein the interim graphs each relate to a different user.

3. The computer-implemented method according to claim 1, wherein the interim graphs are anonymised.

4. The computer-implemented method according to claim 1, wherein the plurality of nodes also includes one or more of a node representing an event identifier, a node representing a time stamp of when the event took place, and a node representing a location of the event.

5. The computer-implemented method according to claim 1, wherein the method further comprises:
receiving a new event including data describing a consultation with one of the plurality of users of a conversation module of the diagnostic system;
encoding the new event using JavaScript Object Notation (JSON);
storing the encoded new event in a queue of events;
decoding and translating the new event into a form compatible with one or more of the plurality of interim graphs; and
adding the translated new event to the interim graph.

6. The computer-implemented method according to claim 5, further comprising:
searching the queue of events for any new events in response to a request to build the user profile; and
in response to identifying a new event in the queue of events, decoding and translating the event into a form compatible with said one or more of the plurality of interim graphs.

7. A computer-implemented method of extracting information concerning a plurality of users, the method comprising:
the method of claim 1,
receiving a query to extract information from the user profile, the information including a plurality of users,
interrogating the user profile to identify a plurality of nodes associated with the plurality of users, and to extract information from nodes linked to the plurality of nodes associated with the plurality of users, and
returning the extracted information for the plurality of users.

8. The computer-implemented method according to claim 7, wherein said information concerning the plurality of users includes one or more of a concept, a location of the event, and a time stamp of the event.

9. The computer-implemented method according to claim 7, wherein the step of returning the extracted information for the plurality of users includes filtering the extracted information to include only information relating to the query.

10. The computer-implemented method according to claim 7, wherein interrogating the user profile to identify a plurality of nodes associated with the plurality of users includes identifying the plurality of nodes within a pre-determined branch factor.

11. A non-transitory computer-readable medium, storing instructions, that when executed by a processor, cause the processor to perform the method according to any preceding claim.
